(19) United States
(12) Patent Application Publication
(10) Pub. No.: US 2010/0185224 A1
(43) Pub. Date: Jul. 22, 2010

(54) SKIN-PIERCING DEVICE FOR TREATMENT OF ACNE

(76) Inventors: Jeffrey M. Wu, Princeton, NJ (US); Ronald J. Gillespie, North Brunswick, NJ (US); Raymond J. Hull, JR., Hampton, NJ (US); Justin E. McDonough, Flemington, NJ (US); Robert A. McLaughlin, Milltown, NJ (US); Nicholas M. Wnek, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/067,883

(22) PCT Filed: Jan. 3, 2008
(86) PCT No.: PCT/US08/50105

§ 371 Date: Mar. 24, 2008

Related U.S. Application Data
(60) Provisional application No. 60/860,703, filed on Nov. 22, 2006.
(60) Provisional application No. 60/896,536, filed on Mar. 23, 2007.

(51) Int. Cl. A61B 17/32

(52) U.S. Cl. 606/182

(57) ABSTRACT

The invention features a skin-piercing device having a piercing element and the use thereof of treating acne.

### BACKGROUND OF INVENTION

Acne is a common skin disease characterized by pimples on the face, chest, and back. It occurs when the pores of he skin become clogged with oil, dead skin cells and bacteria. Inflamed lesions may cause pain, tenderness, itching, or swelling. The most troubling aspects of these lesions are negative cosmetic effects.

Pimple popping has been a pervasive, but sometime impulsive, behavior to quickly reduce the elevation of raised acne, especially pustule type of lesions. The current pimple popping practice is to use fingers to squeeze the pimple, which can carry the risks of infections, post-inflammatory hyperpigmentation, and even scarring. While sterilized hypodermal needles can be used to break the tip of the pimple to release the pus, such needles do not control the depth of and size of cut, which can cause unwanted bleeding and skin damages. Thus, there is clearly a need to provide a safe and hygienic way to pop the pimple and reduce the symptoms of pimple.

The present invention discloses a new method and device to safely assist popping pimple to remove puss and/or treat acne as well as other skin disorders such as cold sores, warts, razor bumps, and age spots.

### SUMMARY OF THE INVENTION

In one aspect, the present invention features a skin-piercing device including: a housing having a skin-contacting surface, wherein the housing includes an opening in the skin-contacting surface; a piercing element disposed within the housing; a forcing assembly urging the piercing element in a direction to project the tip of the piercing element from within the housing through the opening in the skin-contacting surface of the housing; a trigger assembly with a portion contained within the housing arranged to retain the piercing element in a retracted position energizing the forcing assembly and a second portion outside the housing manually actuable to release the piercing element to cause the tip to have a momentary position projecting from the opening; and a fluid-absorbent material affixed to the outside of the housing.

In another aspect, the invention features a method of treating acne, wherein the method includes contacting skin in need of such treatment with the skin-contacting surface of a skin-piercing device including: a housing having a skin-contacting surface, wherein the housing includes an opening in the skin-contacting surface; a piercing element disposed within the housing; a forcing assembly urging the piercing element in a direction to project the tip of the piercing element from within the housing through the opening in the skin-contacting surface of the housing; and a trigger assembly with a portion contained within the housing arranged to retain the piercing element in a retracted position energizing the forcing assembly and a second portion outside the housing manually actuable to release the piercing element to cause the tip to have a momentary position projecting from the opening; wherein the method includes actuating the trigger mechanism to release the piercing element to cause the tip to pierce the skin.

In another aspect, the invention features a kit including a skin-piercing device, the skin-piercing device including: a housing having a skin-contacting surface, wherein the housing includes an opening in the skin-contacting surface; a piercing element disposed within the housing; a forcing assembly urging the piercing element in a direction to project the tip of the piercing element from within the housing through the opening in the skin-contacting surface of the housing; and a trigger assembly with a portion contained within the housing arranged to retain the piercing element in a retracted position energizing the forcing assembly and a second portion outside the housing manually actuable to release the piercing element to cause the tip to have a momentary position projecting from the opening; wherein the kit further includes at least one of the following additional items: (i) a composition including an anti-acne agent, (ii) a re-cocking element to position the piercing element into the retracted position following the release the piercing element, and (iii) an aiming device to assist in positioning the skin-piercing device over the area of skin to be pierced.

Other aspects, features, and advantages of the present invention will be apparent from the detailed description of the invention and from the claims.

### DETAILED DESCRIPTION OF THE DRAWINGS

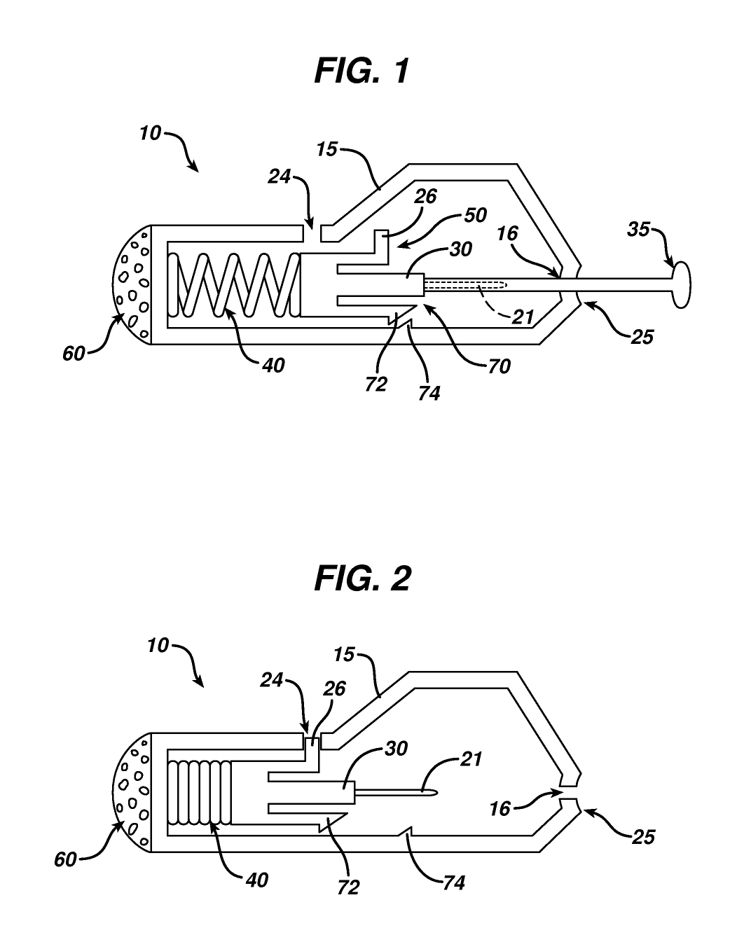

FIG. 1 is a side cross-section view of a skin-piercing device of the present invention in an unused, un-cocked orientation.

FIG. 2 is a side cross-section view of a skin-piercing device of the present invention in a cocked orientation.

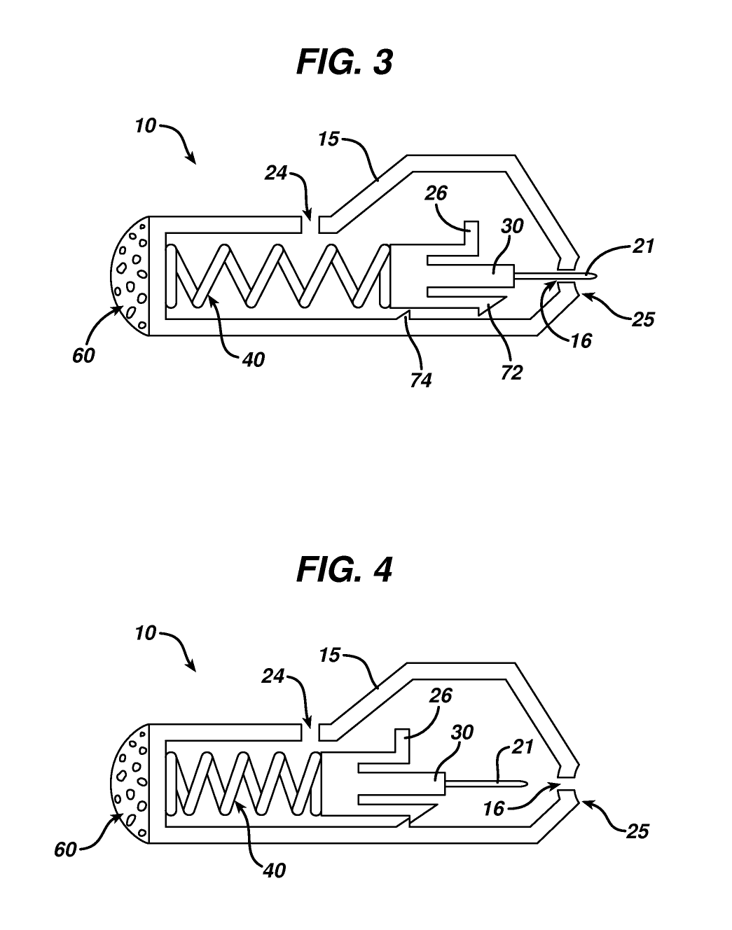

FIG. 3 is a side cross-section view of a skin-piercing device of the present invention in a piercing orientation.

FIG. 4 is a side cross-section view of a skin-piercing device of the present invention in a used orientation.

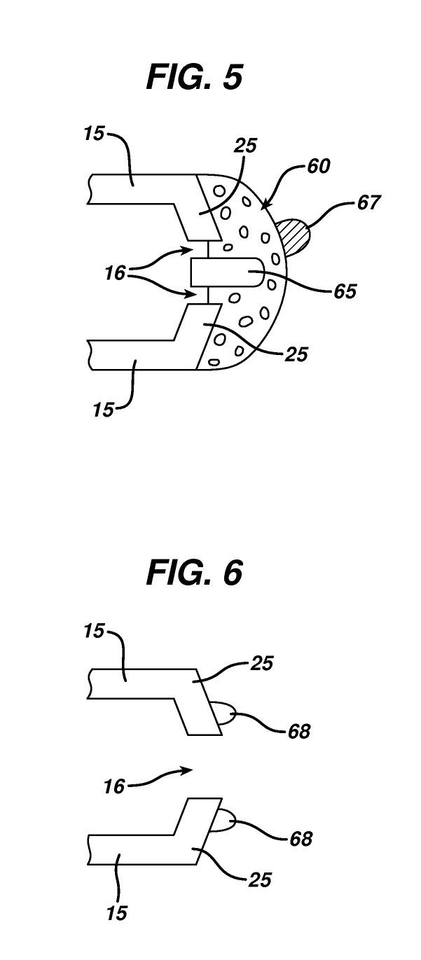

FIG. 5 is a side cross-section view of a portion of a skin-piercing device of the present invention containing a fluid-absorbent material on the skin-contacting surface.

FIG. 6 is a side cross-section view of a portion of a skin-piercing device of the present invention containing a ridge on the skin-contacting surface.

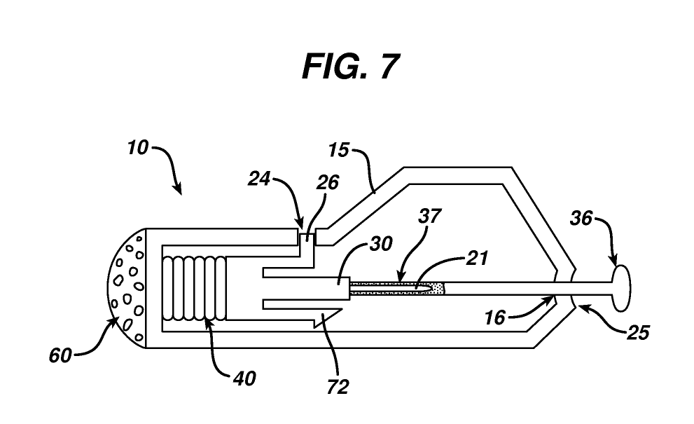

FIG. 7 is a side cross-section view of a skin-piercing device of the present invention that is capable of being repositioned in the cocked orientation.

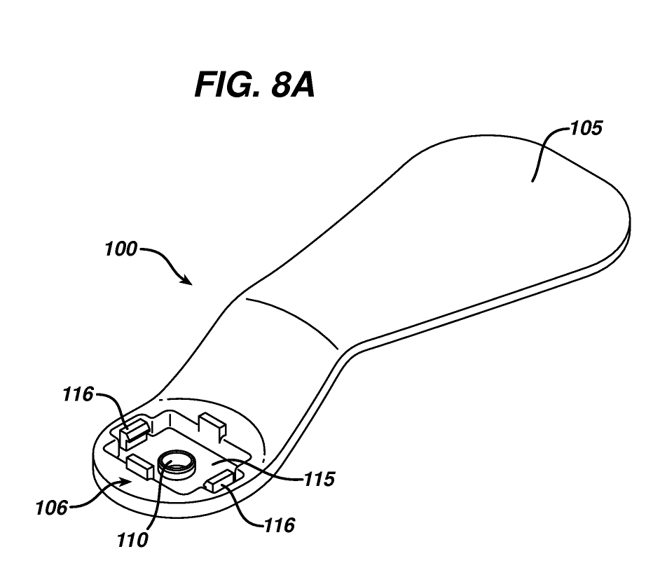

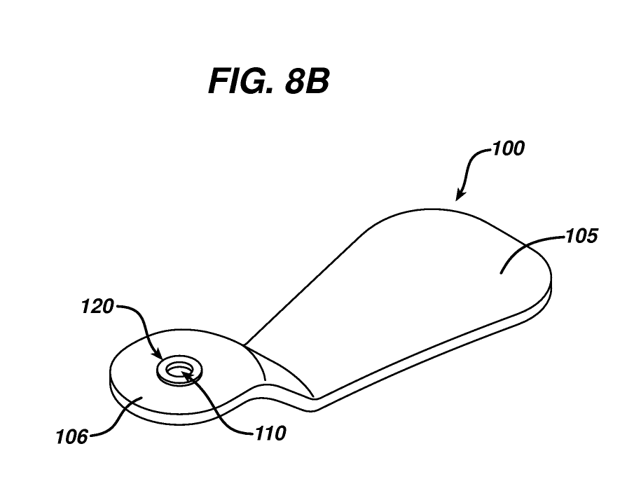

FIG. 8A is a top view of an aiming device of the present invention.

FIG. 8B is a bottom view of an aiming device of the present invention.

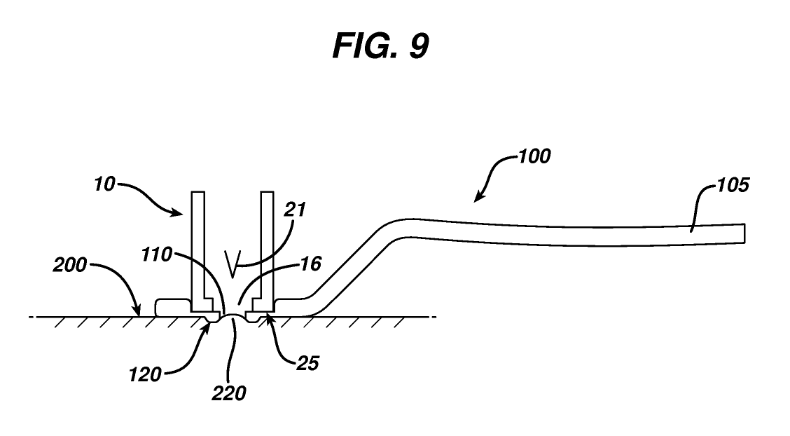

FIG. 9 is a side view of the use of the skin-piercing device and aiming device of the present invention.

### DETAILED DESCRIPTION OF THE INVENTION

It is believed that one skilled in the art can, based upon the description herein, utilize the present invention to its fullest extent. The following specific embodiments can be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified.

In one embodiment, the present invention is directed to a skin-piercing device and the use of that device for treating a skin-condition such as acne. The treatment involves disrupting the stratum corneum of the skin and may or may not further include the application of a composition that permeates into the disrupted skin, such as a composition containing an anti-acne agent. A benefit of such a treatment includes localizing the treatment to a certain area of skin in need of such treatment.

### Definitions

What is meant by a “product” is a product in finished packaged form. In one embodiment, the package is a container such as a plastic, metallic, or cardboard box for storing such skin-piercing device and/or kit. In one embodiment, the product contains instructions directing the user to apply the skin-piercing device to the skin (e.g., for the treatment of a skin condition such as acne).

What is meant by “promoting” is promoting, advertising, or marketing. Examples of promoting include, but are not limited to, written, visual, or verbal statements made on the product or in stores, magazines, newspaper, radio, television, internet, and the like. For promoting the treatment of acne, examples of such statements include, but are not limited to, “treats acne,” “safely pops pimples,” “eliminates acne and/or pimples/blemishes”, and “visibly reduces the symptoms and/or appearance of pimples.” Similar statements can be made for other skin disorders.

The term “treating” or “treatment” of a skin disorder means the treatment (e.g., complete or partial alleviation or elimination of symptoms and/or cure) and/or prevention or inhibition of the skin disorder.

As used herein, “composition” means a composition suitable for administration to the skin.

As used herein, “cosmetically-acceptable” means that the ingredients or compositions which the term describes are suitable for use in contact with the skin without undue toxicity, incompatibility, instability, irritation, allergic response, and the like. This term is not intended to limit the ingredient/composition to which it describes for use solely as a cosmetic (e.g., the ingredient/composition may be a pharmaceutical agent).

As used herein, “safe and effective amount” means an amount of the active agent, compound, carrier, or of the composition sufficient to induce the desired effect, but low enough to avoid serious side effects. The safe and effective amount of the compounds or composition will vary with the area being treated, the age, health and skin/tissue type of the end user, the duration and nature of the treatment, the specific compound or composition employed, the particular cosmetically-acceptable carrier utilized, and like factors.

### Skin-Piercing Device

In one embodiment (as depicted in the FIG. 1), the skin-piercing device of the present invention is a single-use, skin-piercing device 10. Other configurations of single-use lancet devices may also be used, such as those depicted in U.S. Pat. Nos. 5,487,748, 6,258,112, and 6,764,496.

The skin-piercing device 10 includes housing 15. Housing 15 may be made of various rigid materials, including but not limited to plastic and metal. The housing 15 includes two openings, piercing element opening 16 (from which piercing element tip 21 exits the housing 15) on the skin-contacting surface 25 of housing 15 and trigger opening 24 (from which trigger button 26 can be accessed through housing 15).

In one embodiment, the skin-contacting surface 25 has a surface area of less than 50 mm², such as from about 0.5 mm² to about 50 mm², such as from about 1 mm² to about 25 mm². Such a small surface area allows the user to feel where the piercing element tip 21 will exit the skin-piercing device, for example, to position the piercing element opening 16 over the pimple.

In one embodiment, the skin-contacting surface 25 includes a concave shape and piercing element opening 16 is within this concave shape. Such shape allows the user to place the skin-contacting surface, and consequently the piercing element opening 22, over the pimple. In one embodiment, the skin-contacting 25 includes a circumferential ridge 68 (shown in FIG. 6) around piercing element opening 16. Such as ridge can assist the user in positioning the device on the skin surface and/or enhance the vertical pressure on the skin before, during, and after the skin is pierced by the piercing element tip 21, thereby assisting in the removal of the bodily fluids (such as pus from a pimple).

Piercing element 30 includes at least one piercing element tip 21 that pierces the skin following activation of skin-piercing device 10. In one embodiment, piercing element 30 includes from about 2 to about 10 piercing elements tips. Piercing element tip 21 may be made of into shapes, including a needle (either solid or hollow) or a blade. In one embodiment, the piercing element tip is a needle having a gauge of at least 15, such as from about gauge 18 to about gauge 35, such as from about gauge 21 to about gauge 30. Piercing element tip 21 may be made of rigid materials capable of piercing the skin, such as metal. Piercing element 30 is arranged within housing 20, such that it can move from an unused, un-cocked orientation as depicted in FIG. 1, then to a cocked orientation as depicted in FIG. 2, then to a piercing orientation as depicted in FIG. 3 (during which tip 21 temporarily exits the housing 15 through piercing element opening 16), and then to a used orientation as depicted in FIG. 4.

Piercing element 30 preferably moves through housing 15 in a substantially linear direction. In one embodiment, the movement of piercing element 30 through housing 15 is maintained in a linear direction by the inclusion of one or more ridges (not shown) extending from piercing element 30 into one or more linear tracks in the inner wall of housing 15.

Skin-piercing device 10 in preferable obtained by user prior to use in the unused, un-cocked orientation as depicted in FIG. 1, however, it may also be obtained by the user in the cocked orientation. In one embodiment, tip cover 35 extends into the housing 15 through piercing element opening 16 and at least partially covers tip 21. In one embodiment, tip cover 35 serves the purpose of maintaining the sterility of piercing element tip 21.

Tip cover 35 is arranged such that it may be pushed inward into the housing by the user in order to position the piercing element 30 further away from the piercing element opening 16, thereby positioning the piercing element 30 into a cocked orientation as depicted in FIG. 2. Once in this cocked orientation, tip cover 30 may be disengaged from piercing element 30 (thereby uncovering piercing element tip 21) and removed from housing 15.

Skin-piercing device 10 further includes forcing assembly 40 to urge piercing element 30 in a direction to project piercing element tip 21 from within housing 15 through the piercing element opening 16 with sufficient force such that the tip 21 is capable of piercing the skin. Forcing assembly 40 is configured within housing 15 in order to move piercing element 30 from a cocked orientation as depicted in FIG. 2 to a piercing orientation as depicted in FIG. 3 (during which tip 21 temporarily exits the housing 15 through piercing element opening 16). In one embodiment, as depicted in the Figures, the forcing assembly 40 is positioned between housing 15 and the piercing element 30. The forcing assembly 38 may be a spring, as depicted in the Figures. The forcing assembly 38, however, may also be an compressible, elastic material (such a solid silicone material or urethane foam) or a pneumatic or hydraulic assembly.

The skin-piercing device 10 is also preferably arranged such that after the piercing element tip 21 has exited the housing through piercing element opening 16, the piercing element tip 21 tends to be retracted (e.g., by the forcing assembly 38) back into the housing (as depicted in the used orientation of FIG. 4). Such orientation assists in preventing the accidental reuse of the piercing element tip 21.

In one embodiment, the skin-piercing device 10 is adapted such that when piercing element tip 21 exits the housing 20, the piercing element tip 21 has an effective length of less than 1 mm, such as from about 150 microns to about 500 microns, such as from about 150 microns to about 350 microns. What is meant be effective length is the length of the piercing element tip adapted to penetrate into the skin (e.g., the length from the piercing element tip that will penetrate the skin upon activating the skin-piercing device).

Skin-piercing device 10 includes a trigger assembly 50 that serves to both retain the piercing element 30 in a retracted position energizing the forcing assembly 38 and later release piercing element 30 from its cocked orientation. A variety of trigger assemblies can be utilized in the skin-piercing device to serve this purpose. In one embodiment, trigger assembly 50 includes trigger button 26, which is secure to piercing element 30 and is structured to temporarily protrude from housing 15 through trigger opening 24, thereby holding the piercing element 30 in the cocked orientation as depicted in FIG. 2. Other trigger assemblies may also be used, such as one secured to the housing as described in U.S. Pat. No. 5,487,748. In one embodiment (not shown), the trigger assembly is secured to the piercing element and is also used to position the piercing element into the cocked orientation.

The piercing element 30 may be moved by the forcing assembly 38 from the cocked orientation as depicted in FIG. 2 to a piercing orientation as depicted in FIG. 3 by pushing the trigger button 26 back through trigger opening 24 and into the housing 15.

In one embodiment, skin-piercing device 10 further includes restrictor assembly 70, which has the purpose of inhibiting piercing element 30 from being repositioned in the cocked orientation once it has moved into the used orientation. In one embodiment, restrictor assembly 70 includes piercing element protrusion 72 (secured to piercing element 30) and housing protrusion 74 (secured to housing 20). The restrictor assembly 70 is arranged such that piercing element protrusion 72 may pass over housing protrusion 74 when piercing element 30 moves from the cocked orientation as depicted in FIG. 2 to a piercing orientation as depicted in FIG. 3. However, once piercing element 30 has moved from a piercing orientation as depicted in FIG. 3 (during which tip 21 temporarily exits the housing 15 through piercing element opening 16) to the used orientation as depicted in FIG. 4, housing protrusion 74 inhibits the movement of piercing element 30 into the cocked orientation by contacting piercing element protrusion 72, as depicted in FIG. 4. Other restrictor assemblies can also be used, such as that disclosed in PCT Patent Application WO02/43591.

As shown in the Figures, piercing element protrusion 72 is structured such that as it moves from the cocked orientation over housing protrusion 74, the sloped arrangements of piercing element protrusion 72 and housing protrusion 64 permit piercing element protrusion to bend toward piercing element 30, thereby allowing piercing element protrusion 72 to pass over housing protrusion 74 as piercing element 30 moves from the cocked orientation as depicted in FIG. 2 to a piercing orientation as depicted in FIG. 3. Piercing element protrusion 72 is also structured such that once in the used orientation, the piercing element protrusion 72 and housing protrusion 74 abut each other, thereby inhibiting the piercing element 30 from being reengaged by the user into the cocked orientation.

In one embodiment, the skin-piercing device of the present invention includes fluid-absorbent material 60. Absorbent material 60 allows the skin-piercing device 10 to collect bodily fluids such as pus from a pimple pierced by piercing element tip 34. The fluid-absorbent material may be secured to the skin-contacting surface of the skin-piercing device, or to another surface of the skin-piercing device (such as the opposite end of the skin-piercing device as depicted in the Figures).

In one embodiment, the surface of the fluid-absorbent material includes a guide structure 67 having a diameter of from about 0.5 mm to about 3 mm. In one embodiment, the structure is harder than the fluid-absorbent material. Such structure assists the user in position the fluid-absorbent material on the pimple to be treated.

In one embodiment, the fluid-absorbent material may also be used to administer a topical fluid composition (e.g., containing an anti-acne agent as discussed below to the skin in need of such treatment. The skin-piercing device may be packaged such that a composition is (i) added to the fluid-absorbent material proximate to use or (ii) contained within the fluid-absorbent material during storage.

What is meant by fluid-absorbent material is a material that is capable of absorbing fluids in an amount of at least 25 percent of its weight. Examples of absorbent, compressible materials include, but are not limited to, woven and nonwoven materials, hydrogels, hydrocolloids, silicone rubbers, celluloses (e.g., cotton and rayon or their derivatives), wool, polyamides (e.g., nylon), and silk.

In one embodiment (depicted in FIG. 5), fluid-absorbent material 60 encases a reservoir 65 that contains a fluid composition that is expelled from the reservoir 65 upon puncture of the fluid-absorbent material 60 by the piercing element tip. In one embodiment, the fluid composition contains an anti-acne active.

### Recockable Skin-Piercing Device

In one embodiment, the piercing element 30 may be repositioned in the cocked orientation once it has moved into the used orientation. As shown in the embodiment of FIG. 7, skin-piercing device 10 does not include housing protrusion 74. Thus, by inserting re-cocking element 36 into skin-piercing device 10, piercing element 30 may be reposition into the cocked position following use of skin-piercing device 10. In one embodiment, sterilizing section 37 of re-cocking element 36 engages piercing element 30. Sterilizing section 37 may comprise an absorbent material (such as a woven material, non-woven material, or porous urethane foam) that further contains a antimicrobial agent, such as hydrogen peroxide, guanidinium thiocyanate, sodium hydroxide, alcohol (such as ethanol or propanol), benzalkonium chloride, benzethonium chloride, methyl benzethonium chloride, lauric argainate, sugarquat, cetylpyridiunium chloride, and 2,4,4′-trichloro-2-hydroxy diphenylether. The antimicrobial agent provides that when sterilizing section 37 contacts piercing element tip 21, it cleans piercing element tip 21 of any residual blood, pus, or other fluids from its prior use.

### Multiple-Use Skin-Piercing Device

In one embodiment, the skin-piercing device includes multiple piercing elements. In one embodiment, the skin-piercing device is adapted such that each piercing element may only be used once. Examples of such a skin-piercing device include the ACCU-CHEK® Multiclix Lancet Device (Roche Diagnostics, Basel, Switzerland) and devices set forth U.S. Patent Application No. 2004/0260325. In one embodiment, the device is adapted such that the piercing elements can be replaced after use (e.g., disposable storage case can containing multiple piercing element such as that described in European Patent Application No. 638465).

### Aiming Device

In one embodiment, the user utilizes an aiming device to assist in positioning the skin-piercing device over the area of skin to be pierced (e.g., the pimple). In one embodiment, the aiming device is first positioned over the area of skin, following which the skin-piercing device is then secured to such aiming device such that upon activation, the skin-piercing device pierces that desired area of skin.

An example of such an aiming device is set forth in FIGS. 8A and 8B. As set forth in FIG. 8A, the aiming device 100 has a handle portion 105 and a skin-contacting portion 106. The top of skin-contacting portion 106, which is adapted to secure the device to the aiming device 100, has a device-contacting recess 115 that includes securing tabs 116 and orifice 110. In one embodiment, the aiming device 100 is made of a transparent material, such as plastic, such that user can determine that the orifice is positioned in the desired location (e.g., over the pimple to be treated).

As shown in FIG. 8b, the bottom of skin-contacting portion 106 is adapted to contact the skin, and includes orifice 110 within ring 120. By pressing ring 120 against the pimple, the ring 120 applies force on the sides of the pimple, thereby assisting in the removal of pus from the pimple.

FIG. 9 is a depiction of the aiming device 100 being used in conjunction with skin-piercing device 10. Aiming device 100 is first pressed on the surface of skin 200 such that ring 120, and consequently orifice 110, is positioned over pimple 220. Skin-contacting surface 25 of skin-piercing device 10 is then positioned within device-contacting recess 115 and secured with securing tabs 116 (not shown) such that the piercing element opening 16 is positioned over orifice 110 and consequently over pimple 220. Upon activation of skin-piercing device 10, piercing element tip 21 exits skin-piercing device 10 and enters pimple 220.

### Topical Compositions

In one embodiment, the skin-piercing device includes, or is used in conjunction with a topical composition. The composition may be solid, semisolid, liquid, or any combination thereof In particular, examples of solid compositions include but are not limited to bars, sticks, powders, masks, and patches. Examples of semisolid compositions include but are not limited to creams, lotions, gels, ointments, hydrogels, hydrocolloids, foams, mousses, emulsions, and micro-emulsions. Examples of liquid compositions include but are not limited to liquid cleansers, toners, serums, sprays, and aerosols.

In one embodiment, the topical composition includes an anti-acne agent. What is meant by an anti-acne agent is an compound that has been approved by the U.S. Food and Drug Administration for the topical treatment of acne. Examples of anti-acne agents include, but are not limited to, salicylic acid, benzoyl peroxide, sulphur, retinoic acid, isotretinoin, candida bombicola/glucose/methyl rapeseedate ferment, peat water, resorcinol, silt, peat, permethin, azelaic acid, tazorotene, clindamycin, adapalene, erythromycin, sodium sulfacetamide, and combinations thereof In one embodiment, the amount of anti-acne agent in the composition is from about 0.01% to about 10%, for example from about 0.1% to about 5%, or from about 0.5% to about 2% by weight, based on the total weight of the composition.

In one embodiment, the topical composition includes an active agent for the treatment of warts, age spots, razor bumps. Examples of such active agents include ascorbic acid, salicylic acid, and salts and esters thereof

In one embodiment, the active agent and/or composition is applied to the skin proximate to the time of the piercing of the skin with the skin-piercing device (e.g., within about an hour before or after the piercing, such as within about fifteen minutes or within about five minutes).

### Pre-Softening Treatment

In one embodiment, the skin is softened prior to piercing, thereby allowing the skin to be more easily pierced and/or fluids (such as pus) to be more easily removed. Examples of such pre-softening treatments include application of an occlusive or semi-occlusive dressing to the skin piercing site (such as hydrocolloids, hydrogels and transparent film dressings) or hydrating the site (such as cleansing with warm water). Other pre-softening treatments include warming the skin site (e.g., to a temperature from about 35-50° C.) or the application of skin softening agents such as enzymes or acids (e.g., hydroxyl acids).

### Kit

In one embodiment, skin-piercing device(s) of the present invention and optionally companion item(s) are packaged together and marketed as a kit. The examples of the items in the kit may include, but are not limited to, one or more of (i) the skin-piercing devices, (ii) topical compositions in a suitable container/dispenser (such as a tube, a bottle, a pump, a jar, a dropper, a or unit-dose dispenser) to be used before, during, or after the stratum-corneum piercing skin-piercing device application, (iii) aiming devices, and/or (iv) re-cocking elements. Additionally, the kit may also contain a cleansing product to be used to sanitize/sterilize the skin prior to the skin-piercing device application. The kit may also include a film forming composition or bandage to be used after treatment to protect the treated skin site, further assist in the removal of pus, and/or to enhance the therapeutic efficacies for the treated skin.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A skin-piercing device comprising:
a housing having a skin-contacting surface, wherein said housing comprises an opening in said skin-contacting surface,
a piercing element disposed within said housing,
a forcing assembly urging the piercing element in a direction to project the tip of said piercing element from within said housing through said opening in said skin-contacting surface of said housing,
a trigger assembly with a portion contained within said housing arranged to retain said piercing element in a retracted position energizing the forcing assembly and a second portion outside said housing manually actuable to release said piercing element to cause said tip to have a momentary position projecting from said opening, and
a fluid-absorbent material affixed to the outside of said housing.

2. A skin-piercing device of claim 1, wherein said skin-piercing device further comprises a restrictor assembly structured to substantially prevent said piercing element from moving completely into said retracted position after said tip to has projected from said opening.

3. A skin-piercing device of claim 1, wherein said forcing assembly is a spring.

4. A skin-piercing device of claim 1, wherein the surface area of said skin-contacting surface is from about 0.5 mm2 to about 25 mm2.

5. A skin-piercing device of claim 1, wherein said skin-contacting surface comprises a concave shape and said opening is within said concave shape.

6. A skin-piercing device of claim 1, wherein the surface of said fluid-absorbent material comprises a structure having a diameter of from about 0.5 mm to about 3 mm.

7. A skin-piercing device of claim 1, wherein said fluid absorbent material comprises an anti-acne agent.

8. A skin-piercing device of claim 6, wherein said anti-acne agent is selected from the group consisting of salicylic acid and benzoyl peroxide.

9. A skin-piercing device of claim 1, wherein said skin-piercing device is adapted such that the effective length of the tip of the piercing element that projects from the skin-piercing device that is from about 100 to about 500 microns.

10. A skin-piercing device of claim 1, wherein said fluid-absorbent material covers said opening and said fluid absorbent material is capable of being pierced by said piercing element tip.

11. A method of treating acne, wherein said method comprises contacting skin in need of such treatment with the skin-contacting surface of a skin-piercing device including
a housing having a skin-contacting surface, wherein said housing comprises an opening in said skin-contacting surface,
a piercing element disposed within said housing,
a forcing assembly urging the piercing element in a direction to project the tip of said piercing element from within said housing through said opening in said skin-contacting surface of said housing, and
a trigger assembly with a portion contained within said housing arranged to retain said piercing element in a retracted position energizing the forcing assembly and a second portion outside said housing manually actuable to release said piercing element to cause said tip to have a momentary position projecting from said opening,
wherein said method comprises actuating said trigger mechanism to release said piercing element to cause said tip to pierce said skin.

12. A method of claim 11, wherein said skin-piercing device further comprises a fluid-absorbent material affixed to the outside of said housing, and said method comprises absorbing bodily fluids from said pierced skin within said fluid-absorbent material.

13. A method of claim 11, wherein said skin-piercing device further comprises a restrictor assembly structured to substantially prevent said piercing element from moving completely into said retracted position after said tip to has projected from said opening.

14. A method of claim 11, wherein following the release said piercing element said method further comprises repositioning said piercing element into said retracted position and re-actuating said trigger mechanism to release said piercing element to cause said tip to pierce said skin.

15. A method of claim 11, wherein said method further comprises applying to said skin an anti-acne active agent proximate to the time of said piercing the skin with said piercing element.

16. A method of claim 11, wherein said method comprises first positioning a n aiming device over said skin, following which the skin-piercing device is secured to said aiming device prior to actuating said trigger mechanism.

17. A kit comprising a skin-piercing device, said skin-piercing device comprising:
a housing having a skin-contacting surface, wherein said housing comprises an opening in said skin-contacting surface,
a piercing element disposed within said housing,
a forcing assembly urging the piercing element in a direction to project the tip of said piercing element from within said housing through said opening in said skin-contacting surface of said housing, and
a trigger assembly with a portion contained within said housing arranged to retain said piercing element in a retracted position energizing the forcing assembly and a second portion outside said housing manually actuable to release said piercing element to cause said tip to have a momentary position projecting from said opening,
wherein said kit further comprises at least one item selected from the following group of items: (i) a composition comprising an anti-acne agent, (ii) a re-cocking element to position said piercing element into said retracted position following the release said piercing element, and (iii) an aiming device to assist in positioning the skin-piercing device over the area of skin to be pierced.

18. A kit of claim 17, wherein said kit further comprises at least one of said compositions, wherein said anti-acne agent is selected from the group consisting of salicylic acid, benzoyl peroxide, retinoic acid, azelaic acid, tazorotene, clindamycin, adapalene, erythromycin, sodium sulfacetamide, and combinations thereof

19. A kit of claim 17, wherein said kit further comprises at lest one of said re-cocking elements.

20. A kit of claim 17, wherein said kit further comprises at least one said aiming devices.

21. A kit of claim 17, wherein said skin-piercing device has a fluid-absorbent material affixed to the outside of said housing.
